(19) United States
(12) Patent Application Publication
(10) Pub. No.: US 2007/0042976 A1
(43) Pub. Date: Feb. 22, 2007

(54) Method of treating cosmetic and dermatologic conditions by a demethylating agent

(76) Inventor: Gideon Strassmann, Naples, FL (US)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 11/500,476

(22) Filed: Aug. 8, 2006

Related U.S. Application Data
(60) Provisional application No. 60/709,812, filed on Aug. 22, 2005.

(51) Int. Cl. A61K 31/7072; A61K 31/53

(52) U.S. Cl. 514/43; 514/242

(57) ABSTRACT

A method for the treatment of scars and keloids is disclosed. The treatment involves an effective dose of the demethylating agents, 5-azacytidine and 2-deoxy-5-azacytidine. The method also discloses treating skin cancer with a combination therapy comprised of UV radiation and topical application or administration of the above mentioned demethylating agents.

### CROSS REFERENCE TO RELATED APPLICATIONS

This application claims the benefit of U.S. Ser. No. 60/709,812, filed on Aug. 22, 2005, which is incorporated in its entirety herein by reference.

### BACKGROUND OF THE INVENTION

Scars result when the skin repairs wounds caused by an accident, disease, or surgery. Scars are natural part of the healing process. The more the skin is damaged and the longer it takes to heal, the greater the chance of a noticeable scar. The way a scar forms is affected by an individual's age and the location on the body or face. Younger skin makes strong repairs and tends to over-heal, resulting in larger, thicker scars than does older skin. The skin surrounding the jawbone is tighter than the skin on the cheek, making a scar more visible. If a scar is indented or raised, irregular shadows will be seen, giving the skin an uneven appearance. Any one, or a combination, of these factors may result in a scar that, although healthy, may be cosmetically improved by dermatological treatments. None of the currently available treatments are able to remove the scar entirely. These treatments include surgical scar revision, which is a method of removing a scar and rejoining the normal skin in a less obvious fashion and dermabrasion, which is a method of treating acne scars, pockmarks, some surgical scars, or minor irregularities of the skin's surface using an electrical machine to remove the top layers of skin to give a more even contour to the surface of the skin. While it can offer improvement for certain scars, it cannot remove the scar entirely.

Keloids are raised overgrowths of tissue that occur at the site of a skin injury. They occur where trauma, surgery, blisters, vaccinations, acne or body piercing have injured the skin. Less commonly, keloids may form in places where the skin has not had a visible injury. Keloids differ from normal mature scars in composition and size. Some people are prone to keloid formation, which are more common in African-Americans than in other racial groups.

Keloids may be removed with conventional surgery. However, keloids re-appear in more than 45% of patients who have undergone surgical removal, and those that re-appear may be even larger than the original keloid. Keloids are less prone to return if surgical removal is combined with other treatments such as corticosteroid injections. Injection of triamcinolone acetonide, and other corticosteroids, is typically repeated at intervals of four to six weeks. Cryosurgery, which involves freezing the keloid with liquid nitrogen every 20 to 30 days is also used to remove keloids. However, cryosurgery often lightens skin color, limiting the usefulness of this treatment.

The excessive or unregulated production of collagen contributes significantly to scarring and to keloid formation (see Uitto et al., Proc. Natl. Acad. Sci. USA. 82: 5935-5939 (1995); McCauley et al., J. Clin. Immunol. 12: 300-308 (1992)). The process involves TGF β which activates fibroblasts and up-regulates collagen gene expression and secretion (see Peltonen et al., J. Invest. Dermatol. 97: 240-248 (1991)). Moreover, the neutralization of TGF β by anti-TGF β antibody inhibits scar formation (see Shah et al., Lancet 339: 213-214, (1992)). Identification of TGF β modulators provides a novel avenue for therapeutic manipulation of TGF-β action to regulate the repair process (see Roberts et al., Proc. Natl. Acad. Sci. 83: 4167-4171 (1986)); Sporn et al., Science 233: 532-534 (1986).

“Epigenetics”, a term coined in the 1940s, has now evolved to mean heritable changes in gene expression that do not involve changes in DNA sequence (see Holliday et al., Nature 1942; 150:563-565 (1942)). Studies of the molecular basis of epigenetics have largely focused on mechanisms such as DNA methylation and chromatin modifications (Egger G. et al., 429: 457-463 (2004)). DNA methylation is a biochemical modification that, in human cells, primarily affects cytosines when they are part of the symmetrical dinucleotide CpG. It is hypothesized that DNA methylation originally evolved to silence repetitive elements, and that this silencing property has also been put to use in other situations where transcriptional silencing is required, such as imprinting (a process whereby one of the two alleles of a gene are permanently inactivated, depending on from which parent that allele was inherited) and X-chromosome inactivation. Methylation triggers the binding of methylated DNA-specific binding proteins to CpG sites, attracting histone-modifying enzymes that, in turn, focally establish a silenced chromatin state.

Specific DNA methylation inhibitors, such as 5-Azacytidine are currently being used as anti-cancer agents in the USA. Another DNA methylation inhibitor, Decitabine proved to be clinically effective in myelodysplastic syndrome and myeloid leukemias (see Santini et al., Nat. Rev. Cancer; 4: 988-993 (2004)); Issa et al., Curr. Opin. Oncol. 15: 446-451 (2003)). The use of methylation inhibitors in treating cancer revives interest in the epigenetic processes involved in neoplastic development and progression. Further, the potential reversibility of epigenetic changes through pharmacological manipulation makes this area important in cancer management.

Several investigators studied the effect of 5-azacytidine on the regulation of various subtypes of the collagen gene. In transformed rat liver cells and in teratocarcinoma F9 cells, 5-azacytidine increases transcription of the collagen type IV gene and induces transcription of alpha 2 collagen gene, respectively (see Burbelo et al., J. Biol. Chem. 265: 4839-4843 (1990); Chiang et al., J. Biol. Chem. 267: 4988-4991 (1992)). In human rhabdomyosarcoma cells, treatment with 5-azacytidine resulted in transcriptional activation of the Pro collagen alpha I gene. In vitro methylation of the promoter and enhancer of pro alpha I collagen gene leads to its transcriptional inactivation (see Smith et al., J. Biol. Chem. 265: 4839-4843(1990)).TGF β activated collagen induction in resistant cells such as HaCaT. Hela and HepG2. The alpha 2 collagen gene was reactivated by 5-azacytidine (see Yamane et al., J. Cell Physiol. 202: 822-830(2005)). In several other human cancer cells, including fibrosarcomas and breast carcinoma, 5-azacytidine and 5-aza-2-deoxycytidine increased or reactivated collagen gene expression (see Sengupta et al., J. Biol. Chem. 280: 21004-21014(2005)).; Sengupta et al., Cancer Res. 63: 1789-1797(2003)).

These studies were performed primarily on transformed cells or cancer cells, and they are consistent with the conclusion that 5-azacytidine demethylates the promoter region of the collagen gene, which in turn results in the reactivation of the gene and up-regulation of its transcription.

### SUMMARY OF THE INVENTION

This invention relates, in one embodiment, to a method of treating a dermatological or a cosmetic condition in a subject, the method comprising administering to a subject a demethylating agent.

In another embodiment, this invention provides a method of treating a cancerous or precancerous skin lesion in a subject, the method comprising the steps of administering a demethylating agent to a subject; and exposing a cancerous or precancerous skin lesion to UV radiation.

In another embodiment, this invention provides a method of inhibiting TGF β mediated organ fibrosis in a subject comprising administering to a subject a demethylating agent.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, the present invention describes a method of treating a dermatological or a cosmetic condition in a subject comprising administering to a subject a demethylating agent. In some embodiments, dermatological or cosmetic conditions comprise scars, keloids, genital warts, psoriasis, pemphigus, skin blemishes, or other autoimmune skin disorders. In some embodiments, blisters, moles, freckles, hemangiomas, facial spider veins, rosacea, wrinkles/lines, acne, actinic keratosis, angioma, athlete's foot, aquagenic pruritus, atopic dermatitis, baldness, bed sore, Behcet's disease, blepharitis, boil, Bowen's disease, bullous pemphigoid, canker sore, carbuncles, cellulitis, chloracne, chronic dermatitis of the hands and feet, dyshidrosis, cold sores, contact dermatitis, creeping eruption, dandruff, dermatitis, dermatitis herpetiformis, dermatofibroma, diaper rash, eczema, epidermolysis bullosa, erysipelas, erythroderma, friction blister, genital wart, hidradenitis, suppurativa, hives, hyperhidrosis, ichthyosis, impetigo, jock itch, kaposi's sarcoma, keratoacanthoma, keratosis pilaris, lice infection, lichen planus, lichen simplex chronicus, lipoma, lymphadenitis, melasma, miliaria, molluscum contagiosum, nummular dermatitis, Paget's disease of the nipple, pediculosis, pemphigus, perioral dernatitis, photoallergy, photosensitivity, pityriasis rosea, pityriasis rubra pilaris, Raynaud's disease, ring worm, scabies, scleroderma, sebaceous cyst, seborrheic keratosis, seborrhoeic dermatitis, shingles, skin tags, spider veins, squamous cell carcinoma, stasis dermatitis, tick bite, tinea barbae, tinea capitis, tinea corporis, tinea cruris, tinea pedis, tinea unguium, tinea versicolor, tinea, tungiasis, or vitiligo can be treated according to the methods of the present invention. In some embodiments, dermatological conditions that are refractory to steroid treatment can be treated according to the methods of the present invention.

In one embodiment, this invention provides a method of treating a subject suffering from a wound, or reducing the incidence of, or mitigating the severity of, or enhancing or hastening healing of a wound in a subject. In one embodiment, this invention provides a method of treating a subject suffering from a burn, or reducing the incidence of, or mitigating the severity of, or enhancing or hastening the healing of a burn in a subject, the method comprising the step of administering a demethylating agent to a subject.

The term “skin” comprises the epidermal layer of the skin and, in some cases, the dermal layer of the skin. In one embodiment, the epidermal layer of the skin is the outer (epithelial) layer and the stratum corneum, and the deeper connective tissue layer of the skin is called the dermis.

In some embodiments, the invention permits the direct application of demethylating agents to the site where it is needed. In the practice of the invention, it is contemplated that virtually any demethylating agent (i.e., an agent that either promotes demethylation of inhibits methylation) can be employed.

In some embodiments, scar formation is inhibited by direct application of demethylating agents to the injured site. In some embodiments, direct application of demethylating agents comprises topical applications. In some embodiments the topical application of demethylating agents inhibits scar formation. In one embodiment, lingering signs of damage or injury to the skin are inhibited by topically administering demethylating agents.

In some embodiments, over-production of collagen in human skin is inhibited by direct application of demethylating agents. Collagen deposition, in some embodiments, results from injured tissue repair process is inhibited by direct application of demethylating agents. Collagen, in some embodiments has a direct effect on scarring. Hence, in some embodiments, inhibition of over-production of collagen in human skin results in inhibition of scar formation.

Over-production of collagen may result in some embodiments, in lung fibrosis, liver fibrosis, or atherosclerotic heart disease. In some embodiments, treating lung fibrosis, liver fibrosis, scleroderma, Peyronie's disease, or atherosclerotic heart disease comprises inhibiting over-production of collagen via administration of demethylating agents.

In one embodiment, the demethylating agent is 5-azacytidine, 5-aza-2-deoxycytidine, zebularine, procaine, epigallocatechin-3-gallate, RG108, 1-β-D-arabinofuranosyl-5-azacytosine, dihydro-5-azacytidine, L-ethionine, or a combination thereof. In a preferred embodiment, 5-azacytidine, 5-aza-2-deoxycytidine, or a combination thereof is utilized. Another class of demethylating agent, in one embodiment, is the antisense oligonucleotide MG98 directed against the 3′ untranslated region of DNMT1 mRNA. DNMT1 MRNA, in one embodiment, codes for the enzyme DNA methyltransferase 1 responsible for maintenance of DNA methylation.

In some embodiments, the method comprises administering 5-azacytidine, 5-aza-2-deoxycytidine, or a combination thereof. In some embodiments, 5-Azacytidine or 5-aza-2′-deoxycytidine is a chemical analogue of the cytosine nucleoside used in DNA and RNA. In one embodiment, cells in the presence of 5-azacytidine incorporate it into DNA during replication and RNA during transcription. According to the method of the present invention, the incorporation of 5-azacytidine into DNA or RNA inhibits methyltransferase thereby causing demethylation in that sequence.

In some embodiments, the method provides administering a demethylating agent subcutaneously or intradermally. In one embodiment, intradermally is within or between the layers of the skin. In one embodiment, intradermal administration comprises injecting a demethylating agent to a subject. The method in some embodiments also comprises administering a demethylating agent by injection into the area of skin to be treated. In some embodiments, the area of skin to be treated is a lesion and/or tumor. As used herein an “injection” comprises at least a demethylating agent of the invention.

In still further embodiments, the invention is directed to a method of direct administration of a demethylating agent to a scar or a keloid. In such embodiments, the method will preferably involve injecting a solubilized demethylating agent to the affected site.

In some embodiments, the demethylating agent is administered at a dosage of from about 1 ng to 500 μg. In some embodiments, the demethylating agent is administered at a dosage of from about 5 ng to 100 μg. In a preferred embodiment, the demethylating agent is administered at a dosage of from about 10 ng to 50 μg. In one embodiment, the demethylating agent is administered at a dosage of from about 100 ng to 5 μg.

In some embodiments, the method comprises a dosing regimen which comprises a single administration per treatment. In some embodiments, the method comprises a dosing regimen which comprises multiple administration per treatment. In some embodiments, the method comprises administering a combination of a demethylating agent of the current invention with other therapeutic agents as known to one skilled in the art.

In one embodiment, the method of the invention comprises administering a demethylating agent via an intradermal patch. The method in some embodiments also comprises administering the patch adjacent to the area of skin to be treated. As used herein a “patch” comprises at least a demethylating agent of the invention and a covering layer, such that, the patch can be placed over the area of skin to be treated. Preferably, the patch is designed to maximize drug delivery through the stratum corneum and into the epidermis or dermis, reduce lag time, promote uniform absorption, and reduce mechanical rub-off.

In some embodiments, the method comprises administering a topical formulation of the demethylating agent to an affected site of skin. In some embodiments, topical administration according to the present invention comprises aerosol, cream, gel, liquid, ointment, paste, powder, shampoo, spray, patch, disk, or dressing.

In some embodiments, the demethylating agent is administered topically at a concentration of from about 0.0001% to 50%. In some embodiments, the demethylating agent is administered topically at a concentration of from about 0.0005% to 20%. In a preferred embodiment, the demethylating agent is administered topically at a concentration of from about 0.001% to 10%. In one embodiment, the demethylating agent is administered topically at a concentration of from about 0.01% to 5%. In preferred embodiments, the demethylating agent is a cream comprising a demethylating agent at a concentration of from about 0.001% to 10%.

In one embodiment, the term “about” refers to variations of ±25%. In one embodiment, the term “about” refers to variations of ±0.2-25%. In one embodiment, the term “about” refers to variations of ±0.1-15%. In one embodiment, the term “about” refers to variations of ±0.1-10%. In one embodiment, the term “about” refers to variations of ±0.1-5%.

In some embodiments, the invention provides a method of treating a cancerous or precancerous skin lesion in a subject, comprising the steps of: (a) administering to a subject a demethylating agent and (b) exposing a cancerous or precancerous skin lesion to UV radiation. In some embodiments, the combination of UV treatment and a demethylating agent of the present invention will involve the administration of the compound to human subjects, such as patients with squamous cells carcinoma, melanoma, basal cell carcinomas, and other skin cancers that are refractory to 5-fluorouricil (FU).

In some embodiments, a precancerous skin lesion is a change in a particular area or areas of skin that carries the risk of becoming skin cancer. In one embodiment, a precancerous skin lesion comprises a preliminary stage of cancer. In some embodiments, a precancerous skin lesion comprises actinic keratosis, atypical or dysplastic nevi, or premalignant lentigos. In some embodiments, precancerous lesions may be caused by any one or more of the following: UV radiation, genetics, or exposure to cancer-causing substances (carcinogens) such as arsenic, tar or x-ray radiation.

In one embodiment, the method comprises exposing a cancerous or precancerous skin lesion to UV radiation. In one embodiment, UV radiation is the range of invisible radiation wavelengths from about 4 nanometers, on the border of the x-ray region, to about 380 nanometers, just beyond the violet wavelength in the visible spectrum. In a preferred embodiment, according to the present invention, UV radiation is applied in a range from about 280 nanometers to about 380 nanometers.

In some embodiments, UV radiation for therapeutic purposes is termed UV phototherapy. In one embodiment, UV phototherapy according to the present invention comprises UVB radiation. In some embodiments, UVB is emitted from high intensity discharge (HID) mercury vapor, mercury/metal halide vapor lamps, low pressure mercury vapor fluorescent lamps, or a combination thereof. In some embodiments, the number and power of the lamps, the lamp to skin distance, and the individual sensitivity of the patient will be determined for a given treatment as known to one skilled in the art. In some embodiments, UV photochemotherapy is UV phototherapy used in conjunction with the oral or topical application of a chemical photosensitizing agent. In one embodiment, the method of UV photochemotherapy comprises treatment which incorporates low pressure mercury vapor, UVA fluorescent lamps, or HID mercury vapor or mercury/metal halide vapor lamps with an added filter to effectively attenuate the UVB radiation.

In one embodiment, the method comprises exposing a subject to UV radiation after administering a demethylating agent. In another embodiment, the method comprises administering a demethylating agent to a subject after exposing the subject to UV radiation. In some embodiments, the interval between UV radiation and administering a demethylating agent is 0.5-24 hours. In some embodiments, the lag between UV radiation and administering a demethylating agent is 0.5-10 hours.

In one embodiment, the present invention is directed to a method of treating a subject by exposing his/her skin to UV radiation followed by the application of a cream containing a demethylating agent. In the practice of the invention, it is contemplated that virtually any demethylating agent can be employed as part of the dual treatment. In a preferred embodiment, the demethylating agent of the dual treatment comprises any demethylating agent as described hereinabove or that is known in the art.

The methods of the present invention, in one embodiment, may be practiced over a time period ranging from about 1-15 times a day for a period of up to 60 days. In one embodiment, the time period ranges from about 1-8 times a day for a period of up to 30 days.

In some embodiments, the method of the present invention comprises topical, subcutaneous, or intradermal administration of a demethylating agent as described hereinabove.

In one embodiment, the present invention provides a method of inhibiting TGF β, mediated organ fibrosis in a subject comprising administering to a subject a demethylating agent. It is an object of the present invention to provide novel methods for treating, ameliorating, suppressing, inhibiting, and/or preventing organ fibrosis.

In some embodiments, fibroblasts are present in one or more organs such as lung, liver, kidney, vascular vessels, pancreas, and skin, and stimulation of stromal cells specific to the individual organs (kidney mesangial cells, pancreatic stellate cells, etc.) by various cytokines such as TGF β lead to abnormal growth and abnormal extracellular matrix synthesis, leading to organ fibrosis.

In one embodiment, liver fibrosis is treated according to the methods of the present invention. Liver fibrosis, in some embodiments, is caused by excess deposition of extracellular matrices such as collagen during the repair of liver tissue when the balance is lost between hepatocyte necrosis triggered by an external factor, such as a virus and alcohol, or an internal factor involving autoimmune abnormality, and liver regeneration to maintain liver functions.

In one embodiment, the method of inhibiting TGF β, mediated organ fibrosis in a subject comprises systemic administration of a demethylating agent.

In one embodiment, the method of inhibiting TGF β mediated organ fibrosis in a subject comprises administering 5-azacytidine, 5-aza-2-deoxycytidine, or a combination thereof in a parenteral formulation (see Wilmut et al., Nature; 419: 583-586 (2002)), in an oral formulation; or in a food or drink product form (see Egger et al., Nature; 429: 457-463 (2004)).

As described above, 5-azacytidine, 5-aza-2-deoxycytidine, or a combination thereof of the invention may be prepared into various pharmaceutical formulations which are presently known or may be developed in the future, for example various dosing forms for oral administration, parenteral (enteral) administration, transdermal administration, and topical. Any methods which are presently known or may be developed in the future can appropriately be used to prepare pharmaceutical formulations of the invention.

Various forms for medical formulations include for example appropriate solid or liquid formulation forms, for example granules, powders, coated tablets, tablets, (micro) capsules, suppositories, syrup, juice, suspensions, emulsions, drops, injection solutions, and formulations for controlled release of 5-azacytidine, 5-aza-2-deoxycytidine, or a combination thereof.

The methods of the present invention may be utilized for any mammalian subject needing the indicated treatment. Mammalian subjects which may be treated according to the methods of the invention include, but are not limited to, human subjects or patients.

In addition, however, the invention may be employed in the treatment of domesticated mammals which are maintained as human companions (e.g., dogs, cats, horses), which have significant commercial value (e.g., dairy cows. beef cattle, sporting animals), which have significant scientific value (e.g., captive or free specimens of endangered species, experimental species such as rats, mice, monkeys, etc), or which otherwise have value.

One of ordinary skill in the medical or veterinary arts is trained to recognize subjects in need for the treatment/preventive methods of this invention. In particular, clinical and non-clinical trials, as well as accumulated experience, relating to the presently disclosed and other methods of treatment, are expected to inform the skilled practitioner in deciding whether a given subject is at risk of a condition, disease or disorder for treatment by the methods of this invention, and whether any particular treatment is best suited to the subject's needs, including treatment according to the present invention.

Prognosis, diagnosis and/or treatment decisions may be based, in some embodiments, upon clinical indications. It is important to note that the methods of treatment of the present invention need not be restricted to subjects presenting with any particular clinical indication, or other particular marker of a disease or disorder.

In one embodiment, the terms “treatment” or “treating” refer to preventative as well as disorder remitative treatments. In one embodiment, the terms “reducing”, “suppressing” or “inhibiting” refer to lessening, delaying or decreasing, and may refer to symptoms or markers associated with the disease, condition, or disorder, as well as the underlying cause of the disease, condition, or disorder. In some embodiments, the terms “treatment”, “treating”, “reducing”, “suppressing”, and “inhibiting” are used interchangeably.

In one embodiment, the term “administering” refers to bringing a subject in contact with a demethylating agent compound of the present invention, which may be accomplished in vitro, which in one embodiment is in a test tube, or in vivo, which in one embodiment is in cells or tissues of living organisms, for example humans, or ex-vivo, which in one embodiment describes implanting pre-treated cells. In one embodiment, the present invention encompasses administering the compounds of the present invention to a subject, through any route, as will be appreciated by one skilled in the art.

### Materials and Methods

Human Fibroblasts Collagen Synthesis Assay

Human fibroblasts were grown in DMEM (10% FCS) in 24-wells plates to approximately 50% confluence. 5-azacytidine (from Sigma) was added for 5 hrs of 37° C. TGF-β1 was added (10 ng/ml) for 2 hours (hrs) at 37° C. Then, L-[U-14C]-proline solution (specific radioactivity 269 mCi/mmol) was added (1.25 μCi/well) and incubated for 20 hrs at 37° C. The cells were then washed with 5 mM proline in PBS and 5 mM proline in 70% methanol and incubated at 40° C. for 20 hrs. Collagenase solution (0.1 mg/ml in buffer 0.05 M Tris, 0.36 mM CaCl₂, pH=7.5) was then added to the cells. After 20 hrs, the collagenase treated cultures were centrifuged and a new collagenase solution was added to the cells. The cultures were incubated with the enzyme for an additional 20 hrs. Proline incorporated into protein and released by collagenase treatment is defined as “collagen”. Incorporation of labeled proline into collagen was evaluated by scintillation counting of supernatants. In parallel, the cultures were checked for vitality using a 3-(4, 5-dimethylthiazol-2-yl)-2, 5-diphenyltetrazolium bromide (MTT) reaction. Each concentration of 5-azacytidine was analyzed (n=6), and the effect of 5-azacytidine on collagen synthesis were expressed as averaged DPM per number of viable cells +/− standard deviation.

Incorporation of Labeled Proline into Pieces of Human Skin

Pieces of human skin (0.3×0.3 cm) were incubated in DMEM (10% FCS) in 24-well plates with 5-azacytidine at the indicated concentration for 5 hrs at 37° C. and then L-[U-C14]-proline solution (specific activity 269 mCi/mmol) was added (2 μCi/ml) for 20 hrs at 37° C. incubation. The skin was washed with 5 mM proline in PBS and with 5 mM proline in cold methanol 70% (0.8 ml/well). Then, collagenase solution (0.1 mg/mi in buffer containing 0.05 M TRIS, 0.36 mM CaCl₂, pH=7.5) was added to the skin for a 20 hrs incubation at 37° C. Incorporation of labeled proline into collagen (collagenase labile counts) was evaluated by scintillation counting of supernatants after the skin was dried and weighed.

Determination of Cell Death in Response to 5-azacytidine in Human Squamous Cell Carcinoma Line

Human squamous cell carcinoma line (SCL-1) cells were grown in DMEM medium with 10% FCS. 5-azacytidine was added at the indicated concentrations after the cells reached 100% confluence. The cultures were incubated for 24 hrs at 37° C. and then washed in Hepes buffer (pH=7.4) with isotonic salt and exposed to UVB radiation. Cell death was determined using MTT [3-(4, 5-dimethylthiazol-2-yl)-2, 5-diphenyltetrazolium bromide] kit obtained from Sigma. Each concentration of the drug was tested in six wells and the degree of inhibition (vs. controls of untreated cultures) was given as an average (% cell death +/−standard deviation).

### EXAMPLES

### Example 1

5-Azacytidine Inhibits Both Basal Level and TGF β Induced Collagen Biosynthesis by Normal Human Fibroblasts.

Human fibroblasts were grown to about 50% confluence and exposed to medium with or without TGF β. As can be seen in Table 1, at 40 hrs post stimulation, TGF β nearly doubled the amount of collagen produced per cell. It was of importance to standardize the collagen produced per cell number because we previously found that 5-azacytidine inhibits the proliferation of these cells in culture. The addition of 5-azacytidine to the cells inhibited the collagen produced in a dose dependent manner. When a concentration of about 25 pg/mi of the drug was used, the amount of collagen synthesized was reduced by approximately 55%. The addition of 5-azacytidine to TGF β stimulated normal human fibroblasts significantly reduced the quantity of collagen synthesis. The drug inhibited in a dose dependent manner the ability of TGF β to upregulate collagen biosynthesis. Table 1 demonstrates that 5-Azacytidine inhibits both basal and TGF β1 induced collagen biosynthesis by normal human fibroblasts.

TABLE 1

| Azacytidine | Collagen (DPM/cell) | Collagen (DPM/cell) |
|---|---|---|
| (μg/ml) | without TGF β 1 | with TGF β 1 |
| 0 | 5267 +/− 1079 | 9945 +/− 602 |
| 1 | 5306 +/− 717 | 8259 +/− 482 |
| 5 | 4015 +/− 706* | 6801 +/− 1359* |
| 25 | 2323 +/− 139* | 5183 +/− 816* |

*P < 0.05 compared to the corresponding control

### Example 2

### 5-Azacytidine Inhibits Collagen Biosynthesis by Human Skin

This example presents a model for the effect of 5-azacytidine on the biosynthesis of collagen in normal human skin under physiological conditions. To this end, normal skin organ cultures were used. Pieces of human skin were incubated in the presence of 5-azacytidine and the collagen synthesized by the skin was quantified by metabolic labeling using C¹⁴-Proline. As can be seen in Table 2, the drug inhibited collagen biosynthesis in a dose dependent manner. The IC50 was determined to be at about 25 μg/ml, which was similar to the IC50 observed in cultured human fibroblasts.

TABLE 2

| Azacytidine | Collagen | |
|---|---|---|
| (ug/ml) | (DPM/mg tissue) | % Inhibition |
| 0 | 333 +/93 | 0 |
| 6.25 | 199 +/− 34* | 40 |
| 25 | 174 +/− 53* | 48 |
| 100 | 92 +/− 13* | 72 |

*P < 0.01

### Example 3

### 5-Azacytidine and Photo Damage Inhibit the Proliferation of Human Squamous Cells in Culture

The human squamous cells (SCL-1) were grown to about 50% confluence. The cultures were treated with minimal amount of 5-azacytidine, UV radiation, or the combination and cell viability was quantified. Table 3 shows that in the absence of UV exposure, 5-azacytidine at 4.8 μg/mi induced cell death at about 29% of the cells. The exposure of the cells to UV, in the absence of the drug induced a cytotoxic effect in about 22% of the cells. Together, the combination of both UV and 5-azacytidine induced cell death at about 50% of the cancer cells. Thus, photo damage and 5-azacytidine exhibited an additive effect on the viability of human squamous cells in culture.

TABLE 3

| Azacytidine | Without UVB | With UV |
|---|---|---|
| (ug/ml) | (% Cell Death) | (% Cell Death) |
| 0 | 0 | 22 +/− 0.1 |
| 0.53 | 2 +/− 0.7* | 19 +/− 0.5 |
| 1.6 | 5 +/− 0.3* | 21 +/− 0.6 |
| 4.8 | 29 +/− 13* | 50 +/− 0.2 |

*alone, 5-Azacytidine induces cell death on SCL cells at an IC50 of 11 μg/ml

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating a dermatological or a cosmetic condition in a subject, the method comprising administering to said subject a demethylating agent.

2. The method of claim 1, wherein said demethylating agent is 5-azacytidine, 5-aza-2-deoxycytidine, or a combination thereof.

3. The method of claim 1, wherein said treating a dermatological or a cosmetic condition comprises inhibiting scar formation.

4. The method of claim 1, wherein said treating a dermatological or a cosmetic condition comprises inhibiting over-production of collagen in human skin.

5. The method of claim 1, wherein said administering comprises subcutaneous or intradermal injection of said demethylating agent to an affected site of a skin in said subject.

6. The method of claim 5, wherein said demethylating agent is injected at a dosage of from about 10 ng to 50 μg.

7. The method of claim 1, wherein said administering comprises topical administration of said demethylating agent to an affected site of a skin in said subject.

8. The method of claim 7, wherein said agent is formulated as a cream or a gel.

9. The method of claim 7, wherein said demethylating agent is administered topically at a concentration of from about 0.001% to 10%.

10. A method of treating a cancerous or precancerous skin lesion in a subject, the method comprising the steps of:
(a) administering to said subject a demethylating agent; and
(b) exposing said cancerous or precancerous skin lesion to UV radiation.

11. The method of claim 10, wherein said demethylating agent is 5-azacytidine, 5-aza-2-deoxycytidine, or a combination thereof.

12. The method of claim 10, wherein said exposing said subject to said UV radiation follows said administering a demethylating agent to said subject.

13. The method of claim 10, wherein said administering a demethylating agent to said subject follows exposing said subject to said UV radiation.

14. The method of claim 10, wherein said administering a demethylating agent is local.

15. The method of claim 10, wherein said administering to said subject comprises a subcutaneous or intradermal injection of said demethylating agent to the affected site of the skin in said subject.

16. The method of claim 10, wherein said demethylating agent is administered at a dosage of from about 10 ng to 50 μg.

17. The method of claim 10, wherein said administering to said subject comprises topical administration of said demethylating agent to an affected site of a skin in said subject.

18. The method of claim 10, wherein said topical administration comprises a cream or a gel based formulation.

19. The method of claim 17, wherein said demethylating agent is administered topically at a concentration of from about 0.001% to 10%.

20. The method of claim 10, wherein, said skin cancer is basal cell carcinoma.

21. The method of claim 10, wherein said skin cancer is squamous cell carcinoma.

22. The method of claim 10, wherein said skin cancer is melanoma.

23. A method of inhibiting TGF β mediated organ fibrosis in a subject comprising administering to a subject a demethylating agent.

24. The method of claim 23, wherein said demethylating agent is 5-azacytidine, 5-aza-2-deoxycytidine, or a combination thereof.

25. The method of claim 23, wherein said administering to said subject comprises systemic administration of said demethylating agent.

26. The method of claim 23, wherein said organ fibrosis is fibrosis of the lungs, liver, kidneys, vascular vessels, pancreas, skin, or a combination thereof.
